(19) United States
(12) Patent Application Publication
(10) Pub. No.: US 2010/0106243 A1
(43) Pub. Date: Apr. 29, 2010

(54) Implant Made of Biocorrodible Iron or Magnesium Alloy

(75) Inventor: Eric Wittchow, Nuernberg (DE)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/580,155

(22) Filed: Oct. 15, 2009

(30) Foreign Application Priority Data
Oct. 29, 2008 (DE) 10 2008 043 277.6

(51) Int. Cl. A61F 2/06; B05D 5/00

(52) U.S. Cl. 623/1.42; 623/1.46; 427/2.25; 427/2.28; 427/2.3

(57) ABSTRACT

The invention relates to an implant made of a biocorrodable metallic material and having a coating composed of or containing a biocorrodable polyphosphate, polyphosphonate, or polyphosphite.

### CROSS REFERENCE TO RELATED APPLICATIONS

The present invention claims benefit of priority to Germany patent application number DE 10 2008 043 227.6, filed on Oct. 29, 2008, the contents of which are herein incorporated by reference in their entirety.

### FIELD OF THE INVENTION

The invention relates to an implant made of a biocorrodable iron or magnesium alloy and having a polymer coating.

### BACKGROUND OF THE INVENTION

Implants have found application in modern medical technology in many different embodiments. They are used, for example, for supporting vessels, hollow organs, and duct systems (endovascular implants), for attaching and temporarily fixing tissue implants and tissue transplants, as well as for orthopedic purposes, for example as pins, plates, or screws.

For example, the implantation of stents has become established as one of the most effective therapeutic measures in the treatment of vascular diseases. Stents perform a support function in hollow organs of a patient. For this purpose, stents of conventional design have a filigreed support structure made of metallic braces, which are initially in a compressed form for insertion into the body, and are then expanded at the site of application. One of the main fields of application of such stents is to permanently or temporarily widen and keep open vascular constrictions, in particular constrictions (stenoses) of the coronary vessels. In addition, aneurysm stents, for example, used for supporting damaged vascular walls are known.

Stents have a circumferential wall of sufficient load capacity to keep the constricted vessel open to the desired extent, and have a tubular base body through which the blood flows through unhindered. The supporting circumferential wall is generally formed by a lattice-like support structure which allows the stent to be inserted in a compressed state, with a small outer diameter, up to the constriction in the particular vessel to be treated, and at that location, for example by use of a balloon catheter, to be expanded until the vessel has the desired enlarged inner diameter. To avoid unnecessary damage to the vessel, there should be little or no elastic return of the stent after the expansion and after the balloon is removed, so that during the expansion the stent need be widened only slightly beyond the desired end diameter. Additional desirable criteria for a stent include, for example, uniform surface coverage and a structure which allows a certain degree of flexibility with respect to to the longitudinal axis of the stent. In practice, to achieve the referenced mechanical properties the stent is generally made of a metallic material.

In addition to the mechanical properties of a stent, the stent should also be made of a biocompatible material to prevent rejection reactions. Stents are currently used in approximately 70% of all percutaneous surgical procedures; however, in-stent restenosis occurs in 25% of all cases due to excessive neointimal growth caused by strong proliferation of the smooth muscle cells of the arteries and a chronic inflammatory reaction. Various approaches are used to reduce the rate of restenosis.

One approach for reducing the rate of restenosis is to provide a pharmaceutically active substance on the stent which counteracts the mechanisms of restenosis and facilitates the healing process. The active substance, in the pure form or embedded in a carrier matrix, is applied as a coating or filled into cavities in the implant. Examples include the active substances sirolimus and paclitaxel.

Another, more promising approach to solving the problem lies in the use of biocorrodable metals and their alloys, since it is usually not necessary for the stent to have a permanent support function. Thus, for example, DE 197 31 021 A1 discloses the production of medical implants from a metallic material whose primary component is iron, zinc, or aluminum, or an element from the group of alkali metals or alkaline earth metals. Alloys based on magnesium, iron, and zinc have been described as particularly suitable. Secondary components of the alloys may be manganese, cobalt, nickel, chromium, copper, cadmium, lead, tin, thorium, zirconium, silver, gold, palladium, platinum, silicone, calcium, lithium, aluminum, zinc, and iron. Also known from DE 102 53 634 A1 is the use of a biocorrodable magnesium alloy containing >90% magnesium, 3.7-5.5% yttrium, 1.5-4.4% rare earth metals, and the remainder <1%, which is particularly suitable for producing an endoprosthesis, for example in the form of a self-expanding or balloon-expandable stent. The use of biocorrodable metallic materials in implants may result in a considerable reduction in rejection or inflammatory reactions.

The frequently acidic products of degradation of known biocorrodable polymers may result in an inflammatory reaction in the surrounding tissue; i.e., the material has only moderate biocompatibility. Thus, for example, it has been demonstrated that for biodegradable polyorthoesters, it is not the polymer itself or the intermediate products during degradation that are responsible for the inflammation, but, rather, the released acetic acid (Zignani et al., Subconjunctival biocompatibility of a viscous bioerodible poly(orthoester), J. Biomed. Mater. Res., 1997, 39 pp. 277-285). Other causative factors for poor biocompatibility, primarily process engineering-related, are also known.

In addition to the undesired biological response to the acidic degradation products in the form of inflammation, in the case of an implant made of a biocorrodable magnesium alloy the change in pH also influences the formation of a passivation layer, which usually greatly retards the degradation of the implant after contact with moisture or blood. If the pH of the passivation layer is lowered by release of acidic degradation products, formation of the hydroxide-containing passivation layer is impaired, thus generally accelerating the degradation. This causes a stent made of a biocorrodable magnesium alloy, for example, to lose its support capacity more quickly. The referenced negative effects have been observed in several of the present applicant's tests in which the combination of polymers with acidic degradation products, such as polyesters (PL A, PLGA, or P4BH), polyanhydrides, or polyester amides, with a stent made of a biocorrodable magnesium alloy was investigated. This generally applies to all polymers whose chain structure results from a chemical reaction of one or more carboxylic acid functional groups of the corresponding monomers.

### BRIEF SUMMARY OF THE INVENTION

The object of the invention is to alleviate or eliminate one or more of the described problems. The invention relates to an implant made of a biocorrodable metallic material and having a coating composed of or containing a biocorrodable polyphosphate, polyphosphonate, or polyphosphite. The invention is based on the finding that the degradation of the referenced polyphosphoesters does not lead to acidic degradation products, since the chain structure does not result from carboxylic acid functional groups. Formation of the passivation layer on the surface of the implant made of a biocorrodable metal layer may be facilitated by using degradable polymers whose degradation products have a neutral or even slightly basic reaction, not an acidic reaction.

### DETAILED DESCRIPTION OF THE INVENTION

Polyphosphoesters are polymers having a linear base structure of covalently bonded monomers which contain a hydrophilic phosphate, amidophosphate, phosphonate, or phosphite group, and a hydrophobic group which links the phosphorus-containing groups in the polymer.

A substituted or unsubstituted alkyl radical may also be bound to the phosphate or phosphonate group. The lipophilicity of the polymer, and therefore the degradation rate, may be influenced by the hydrophobic group, i.e., the alkyl radical. The degradation rate is generally reduced with increasing lipophilicity of the polymer. Polyphosphoesters, in particular poly(alkylene phosphates), exhibit very low cytotoxicity (Wang et al., J. Am. Chem. Soc., 2001, 123, pp. 9480-9481). Poly(alkylene phosphates) may be synthesized by a ring-opening polymerization of five- or six-membered cyclic esters of phosphoric acid and derivatives thereof (Penczek et al., Biomacromolecules, 2005, 6, pp. 547-551). The polymers are generally soluble in alcohols (especially methanol), and may be applied to the implant, for example, via conventional spray methods (possibly in a mixture with an active substance).

The characteristics of the polymer are controlled in a particularly simple manner by leaving the main chain unmodified, and in the last synthesis step binding a suitable substituent to the phosphate or phosphonate group. Although the main chain of the polymer decomposes into neutral diols and phosphate, the characteristics of the polymer may be controlled by varying the substituents:
An amino group on the substituent results in more rapid degradation of the polymer, probably due to an autocatalytic effect and an increase in the pH caused by the amines released during decomposition.
A methyl or ethyl group as substituent noticeably retards the degradation.

When a polyphosphite is activated by reaction with chlorine, in a second step the polyphosphite may be reacted with various nucleophilic substances. These may be amino acids or oligopeptides, for example, resulting in polyamidophosphates.

The substituent may also be used for binding a pharmacologically active substance which is not released until the polymer undergoes degradation. Thus, the substituent may contain a nitro group, for example, which metabolizes in the body with release of NO, resulting in localized, desired vessel dilation. More complex pharmacologically active compounds may also be directly bound to a polyphosphite via the corresponding chloride if the compounds contain a reactive amine functional group or a hydroxy group. Examples of binding of suitable active substances include amlopidine (binding via NH₂), bosentan, paclitaxel, and sirolimus (binding via OH in each case).

The biocorrodable polymer is preferably a poly(alkylene phosphate) of formula (I)

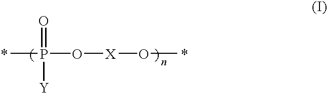

wherein Y stands for
NR₁R₂, where R₁ and R₂ are independently selected to be H or a substituted or unsubstituted C1-C10 alkyl radical;
OR, where R is H or a substituted or unsubstituted C1-C10 alkyl radical; or
an amino acid bound to P via its amine functional group; and
   X is a substituted or unsubstituted ethylene or propylene bridge.

In particular, Y is an amino acid selected from the group lysine, arginine, histidine, alanine, and phenylalanine.

A coating within the meaning of the invention is an application of the components, at least in places, to the base body of the implant, in particular the stent. The entire surface of the base body of the implant/stent is preferably covered by the coating. A layer thickness is preferably in the range of 1 nm to 100 μm, particularly preferably 300 nm to 15 μm. The coating is composed of a biocorrodable polyphosphoester or contains such a polyphosphoester. The percentage of polyphosphoester by weight in the components of the coating forming the carrier matrix is at least 30%, preferably at least 50%, particularly preferably at least 70%. A blend of various polyphosphoesters may be present. The components of the coating include the materials which function as the carrier matrix, i.e., materials which are necessary for the functional properties of the carrier matrix, for example, also auxiliary materials for improving the viscosity characteristics, gel formation, and ease of processing. These components do not include the optionally added active substances or marker materials. The coating is applied directly to the implant surface, or an adhesive layer is applied first. These may be, for example, silanes or phosphonates having a reactive end group (COON, OH, NH₂, aldehyde) applied to the surface of the base material, or an oxidic conversion layer on the base material.

The polyphosphoesters used according to the invention are highly biocompatible and biocorrodable. Processing may be performed according to standard coating methods. Single-layer as well as multilayer systems (for example, so-called base coat, drug coat, or top coat layers) may be produced.

The polymer may act as a carrier matrix for pharmaceutical active substances, X-ray markers, or magnetic resonance markers. For implants made of a biocorrodable metallic material the X-ray marker cannot be directly applied to the product, since it would influence the degradation of the stent by formation of localized elements. On the other hand, in the matrix composed of polyphosphoester the marker is shielded from the base body.

Within the meaning of the invention, a “biocorrodable iron or magnesium alloy” is understood to mean a metallic structure having iron or magnesium as the primary component. The primary component is the alloy component having the highest proportion by weight in the alloy. A proportion of the primary component is preferably greater than 50% by weight, in particular greater than 70% by weight. The biocorrodable magnesium alloy preferably contains yttrium and other rare earth metals, since such an alloy is well suited due to its physical-chemical properties and high biocompatibility, in particular also its degradation products. It is particularly preferred to use a magnesium alloy having a composition of 5.2-9.9% by weight of rare earth metals, of which yttrium constitutes 3.7-5.5% by weight, and the remainder <1% by weight, wherein magnesium makes up the remaining proportion of the alloy to give 100%. This magnesium alloy has been experimentally proven, and its particular suitability, i.e., high biocompatibility, favorable processing characteristics, good mechanical parameters, and corrosion characteristics which are adequate for the intended purpose, has been demonstrated in initial clinical trials. In the present context, the collective term “rare earth metals” refers to scandium (21), yttrium (39), lanthanum (57), and the following 14 elements following lanthanum (57): cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

The compositions of polyphosphoester and the iron or magnesium alloy are selected so that they are biocorrodable. Artificial plasma, as specified under EN ISO 10993-15:2000 for biocorrosion tests (composition: NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L), is used as a test medium for testing the corrosion characteristics of polymer materials or alloys. For this purpose, a sample of the material to be tested is kept at 37° C. in a sealed sample container containing a defined quantity of the test medium. The samples are withdrawn at time intervals corresponding to the anticipated corrosion characteristics, from a few hours to several months, and analyzed in a known manner for signs of corrosion. Artificial plasma according to EN ISO 10993-15:2000 corresponds to a medium that is similar to blood, thus providing an opportunity to reproducibly duplicate the physiological environment within the meaning of the invention.

Implants within the meaning of the invention are devices which are inserted into the body by surgical methods, and include attachment elements for bones, for example screws, plates, or pins, surgical suture material, intestinal clamps, vessel clips, prostheses for hard and soft tissue, and anchoring elements for electrodes, in particular for pacemakers or defibrillators.

The implant is preferably a stent. Stents of conventional design have a filigreed support structure made of metallic braces, which are initially in an unexpanded state for insertion into the body, and are then widened to an expanded state at the site of application. Brittle coating systems are unsuitable due to the manner of use; in contrast, polyphosphoesters have particularly suitable material properties, such as viscosity and flexibility which are adequate for the purpose. The stent may be coated before or after being crimped onto a balloon.

A second aspect of the invention concerns the use of biocorrodable polyphosphoesters as coating material for a stent made of a biocorrodable iron or magnesium alloy.

The invention is explained in greater detail below with reference to one exemplary embodiment.

Substituted polyphosphonates may be prepared from corresponding polyphosphites. The corresponding polyphosphite may be prepared by a ring-opening polymerization, since larger molar masses (Mₙ>10⁵) may be produced than by polycondensation. The preparation is carried out analogously to procedures in the literature (Penczek et al., Makromol. Chem. 1977, 178, pp. 2943-2947):

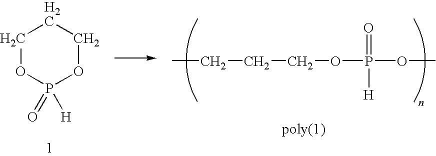

A solution of 7 mol oxyphosphonoyloxytrimethylene (1) and 3×10⁻² mol/L [(i-C₄H₉)₃Al] was reacted in 1000 mL dry THF at 25° C. for 24 hours until equilibrium was reached. The product was precipitated in dry toluene. Poly(oxyphosphonoyloxytrimethylene) (poly(1)) precipitated as a white powder sensitive to hydrolysis, in a yield of 50%.

Dry Cl₂ gas was introduced into a 10% solution of polymer (poly(1)) in dry CH₂Cl₂ at 0° C. until a permanent yellow color was obtained. Excess Cl₂ was then removed under vacuum until a clear solution of poly(alkenyl chlorophosphate) (2) was obtained (procedure analogous to Penczek et al. Macromolecules 1993, 26, pp. 2228-2233). A solution of 215 mol-% benzylamine in CH₂Cl₂ was added to the clear solution of poly(alkenyl chlorophosphate) (2) in CH₂Cl₂ at room temperature over a period of 1 hour. The reaction mixture was stirred for an additional hour at 0° C. whereupon benzylamine hydrochloride precipitated. After filtering off the hydrochloride, a clear solution was obtained which was concentrated under vacuum to 15-20% of its original volume, then the product poly(2-aminobenzylpropylene phosphate) (3) was precipitated from acetonitrile and dried (procedure analogous to Penczek et al., Macromolecules 1986, 19. pp. 2228-2233).

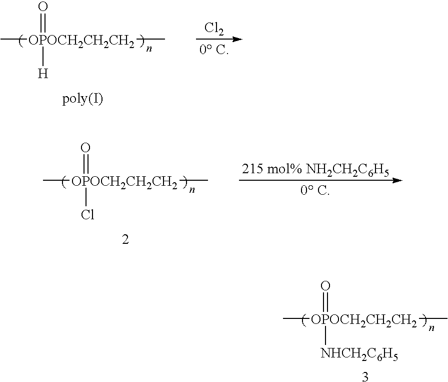

### EXAMPLE

The inventive method and/or the inventive implant is/are explained in the following example. All the features described constitute the subject of the invention, regardless of how they are combined in the claims or their references back to preceding claims.

### Example: Coating of a Stent

A stent made of the biocorrodable magnesium alloy WE43 (4% by weight yttrium, 3% by weight rare earths other than yttrium, with the remainder magnesium and production-related impurities) was coated as follows:

A solution of poly(2-aminobenzylpropylene phosphate) (3) in CH₂Cl₂ (30% by weight) was prepared. Dust and residues were cleaned from the stent, and the stent was clamped in a suitable stent coating apparatus (DES coater, Biotronik in-house development). Using an airbrush system (from EFD or Spraying System), the revolving stent was coated with the solution on one-half side under constant environmental conditions (room temperature, 42% relative humidity). At a nozzle distance of 20 mm, a stent 18 mm in length was coated after approximately 10 minutes. After the intended coating mass was reached, the stent was dried for 5 min at room temperature, and then was rotated and reclamped, and the uncoated side was coated in the same manner. The final coated stent was dried in a vacuum oven at 40° C. for 36 hours (Vakucell: MMM).

The layer thickness of the applied polyphosphoester was approximately 2 to 7 μm.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implant made of a biocorrodable iron or magnesium alloy and having a coating composed of or containing a biocorrodable polyphosphate, polyphosphonate, or polyphosphite.

2. The implant according to claim 1, wherein the implant is a stent.

3. The implant according to claim 1, wherein the coating contains an active substance.

4. The implant according to claim 1, containing a poly(alkylene phosphate) of formula (I) below: 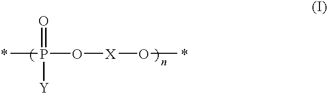
wherein Y stands for
NR₁R₂, where R₁ and R₂ are independently selected to be H or a substituted or unsubstituted C1-C10 alkyl radical;
OR, where R is H or a substituted or unsubstituted C1-C10 alkyl radical; or
an amino acid bound to P via its amine functional group; and
X is a substituted or unsubstituted ethylene or propylene bridge.

5. A method of manufacturing a stent made of biocorrodable metallic material, comprising:
providing a biocorrodable polyphosphate, polyphosphonate, or
polyphosphite as coating material.
